(19) United States
(12) Patent Application Publication
(10) Pub. No.: US 2023/0255497 A1
(43) Pub. Date: Aug. 17, 2023

(54) SYSTEM AND METHODS FOR PHYSIOLOGICAL PARAMETER MEASUREMENT

(71) Applicant: Synapsis Medical Inc., Edmonton (CA)

(72) Inventor: Preetam ANBUKARASU, St. Albert (CA)

(21) Appl. No.: 17/822,404

(22) Filed: Aug. 25, 2022

Related U.S. Application Data
(63) Continuation of application No. PCT/US2021/020316, filed on Mar. 1, 2021.
(60) Provisional application No. 62/984,071, filed on Mar. 2, 2020.

(51) Int. Cl. A61B 5/02; A61B 5/00; A61B 5/0205

(52) U.S. Cl. CPC A61B 5/02007; A61B 5/489; A61B 5/0205; A61B 5/6831; A61B 2560/0223; A61B 2562/0247; A61B 5/02233

(57) ABSTRACT

Provided are systems and methods to evaluate cardiovascular health parameters, comprising a system configured to create a pressure map of the physiological surface (such as, skin) using a combination of actuators and pressure sensing arrays.

### CROSS-REFERENCE

This application is a continuation of PCT Application No. PCT/2021/20316, filed on Mar. 1, 2021, which claims the benefit of U.S. Provisional Application No. 62/984,071, filed Mar. 2, 2020, which application is incorporated herein by reference.

### SUMMARY

Provided herein are embodiments of a physiological measurement system comprising: a support configured to engage a wearer; and a sensor array coupled to the support, the sensor array configured to sense one or more pressure changes on a skin surface of the wearer and determine one or more physiological parameters of the wearer based on the one or more sensed pressure changes.

In some embodiments, the system further comprises a data processing module configured to generate a dynamic pressure map of the skin surface based on the one or more sensed pressure changes. In some embodiments, the system further comprises a biometric display unit to display the dynamic pressure map. In some embodiments, the biometric display unit is physically coupled to the support. In some embodiments, the data processing module is configured to wirelessly transmit the dynamic pressure map to the biometric display unit. In some embodiments, at least one of the one or more sensed pressure changes is associated with a blood vessel proximal to the skin surface of the wearer.

In some embodiments, at least one of the one or more physiological parameters are parameters is associated with the blood vessel. In some embodiments, the one or more physiological parameters comprise a systolic blood pressure, a diastolic blood pressure, a mean blood pressure, a heart rate, a blood vessel viscoelasticity, an arterial stiffness, or a combination thereof.

In some embodiments, the support is configured to engage a portion of a limb of the wearer, the support configurable in: a non-occlusion configuration; and an occlusion configuration for occluding the blood vessel. In some embodiments, the support is further configurable in a partial occlusion configuration for partially occluding the blood vessel. In some embodiments, a mechanism to change the configuration of the support comprises an actuator mechanism, a fluid actuation mechanism, or a combination thereof. In some embodiments, the support comprises a sleeve, wherein the sleeve engages the portion of the limb of the wearer at an effective diameter, and wherein the effective diameter is reduced to occlude the blood vessel. In some embodiments, the system is operable in a calibration mode, wherein operation in the calibration mode configures the support in the occlusion configuration for calibrating baseline parameters the one or more physiological parameters. In some embodiments, the system is operable in a continuous monitoring mode, wherein operation in the calibration mode configures the support in the partial occlusion configuration for monitoring the one or more physiological parameters

In some embodiments, the sensor array is comprised of a plurality of sensing elements. In some embodiments, the plurality of sensing elements comprises thin-film transducers. In some embodiments, the thin-film transducers are polymeric thin-film transducers.

Further provided are embodiments of a method of measuring one or more physiological parameters, comprising: measuring one or more changes in pressure on one or more a regions of a skin surface of a wearer; and generating a dynamic pressure map of the skin surface of the wearer based on the measured one or more changes in pressure of the skin surface.

In some embodiments, measuring one or more changes in pressure on the skin surface of the wearer further comprises measuring physiological reaction forces on the skin surface of the wearer. In some embodiments, the physiological reaction forces are oscillometric pressure waves generated by a blood vessel proximal to the skin surface of the wearer. In some embodiments, the method further comprises a step of determining a location of a blood vessel proximal to the skin surface of the wearer. In some embodiments, the method further comprises isolating one or more changes in pressure of the skin surface at the location of the blood vessel. In some embodiments, the step of isolating the one or more changes in pressure at the location of the blood vessel comprises carrying out a time, frequency, and space domain clustering analysis of a comparison of the changes in pressure at the location of the blood vessel relative to one or more changes in pressure on the skin surface of the wearer at a location away from the blood vessel.

In some embodiments, the method further comprises steps of occluding the blood vessel; and calibrating the one or more measured pressure changes of the skin surface. In some embodiments, the step of occluding the blood vessel comprises fully occluding the blood vessel. In some embodiments, the method further comprises steps of partially occluding the blood vessel; and monitoring for one or more changes in pressure of the skin surface.

In some embodiments, one or more physiological parameters comprise a systolic blood pressure, a diastolic blood pressure, a mean blood pressure, a heart rate, a blood vessel viscoelasticity, an arterial stiffness, or a combination thereof.

Further provided herein are embodiments of a wearable physiological measurement device, comprising: a support configured to engage a portion of a limb of a wearer; a sensor array coupled to the support and configured to sense one or more pressure changes on a skin surface of the wearer; and a data processing module configured to generate a dynamic pressure map of the skin surface based on the one or more sensed pressure changes on the skin surface, wherein one or more physiological parameters of the wearer are derived from the dynamic pressure map.

In some embodiments, the data processing module is configured to transmit the dynamic pressure map to a biometric display unit. In some embodiments, the biometric display unit is physically coupled to the support. In some embodiments, at least one of the one or more sensed pressure changes is associated with a blood vessel proximal to the skin surface of the wearer. In some embodiments, the at least one of the one or more physiological parameters is associated with the blood vessel.

In some embodiments, the support is configurable in a non-occlusion configuration; and an occlusion configuration for occluding the blood vessel. In some embodiments, the support is further configurable in a partial occlusion configuration for partially occluding the blood vessel. In some embodiments, a mechanism to change the configuration of the support comprises an actuator mechanism, a fluid actuation mechanism, or a combination thereof. In some embodiments, the support comprises a sleeve, wherein the sleeve engages the portion of the limb of the wearer at an effective diameter, and wherein the effective diameter is reduced to occlude the blood vessel. In some embodiments, the device is operable in a calibration mode, wherein operation in the calibration mode configures the support in the occlusion configuration for calibrating baseline parameters for the one or more physiological parameters. In some embodiments, the device is operable in a calibration mode, wherein operation in the calibration mode configures the support in the occlusion configuration for monitoring the one or more physiological parameters.

In some embodiments, the sensor array is comprised of a plurality of sensing elements. In some embodiments, the plurality of sensing elements comprises thin-film transducers. In some embodiments, the thin-film transducers are polymeric thin-film transducers. In some embodiments, the one or more physiological parameters comprise a systolic blood pressure, a diastolic blood pressure, a mean blood pressure, a heart rate, a blood vessel viscoelasticity, an arterial stiffness, or a combination thereof.

Further provided herein are embodiments of a wearable blood pressure measurement device, comprising: a sleeve configured to engage a portion of a limb of a wearer; a sensor array coupled to the sleeve and configured to sense one or more changes in pressure on a skin surface associated with a blood vessel; and a data processing module configured to generate a dynamic pressure map of the blood vessel based on the one or more sensed pressure changes, wherein a blood pressure of the wearer is determined from the dynamic pressure map.

In some embodiments, the data processing module is configured to transmit the dynamic pressure map to a biometric display unit. In some embodiments, the biometric display unit is physically coupled to the sleeve. In some embodiments, the sleeve engages the portion of the limb of the wearer at an effective diameter, the sleeve configurable in: a non-occlusion configuration; and an occlusion configuration for occluding the blood vessel. In some embodiments, the sleeve further comprises a partial occlusion configuration for partially occluding the blood vessel. In some embodiments, the effective diameter of the sleeve is reduced to occlude the blood vessel. In some embodiments, a mechanism to reduce the effective diameter of the sleeve comprises an actuator mechanism, a fluid actuation mechanism, or a combination thereof. In some embodiments, the device is operable in a calibration mode, wherein operation in the calibration mode configures the sleeve in the occlusion configuration for calibrating baseline parameters the blood pressure of the wearer. In some embodiments, the device is operable in a continuous monitoring mode, wherein operation in the calibration mode configures the sleeve in the partial occlusion configuration for monitoring the blood pressure of the wearer.

In some embodiments, the sensor array is comprised of a plurality of sensing elements. In some embodiments, the plurality of sensing elements comprises thin-film transducers. In some embodiments, the thin-film transducers are polymeric thin-film transducers. In some embodiments, the blood pressure comprises a systolic blood pressure, a diastolic blood pressure, a mean blood pressure, or a combination thereof. In some embodiments, a heart rate, a blood vessel viscoelasticity, an arterial stiffness, or a combination thereof is further determined from the dynamic pressure map.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a cardiovascular parameter measurement system worn by a user, according to some embodiments;

FIG. 2 depicts a cardiovascular parameter measurement system worn by a user, according to some embodiments;

FIG. 3 depicts a block diagram of modules of a cardiovascular parameter measurement system, according to some embodiments;

FIG. 4a depicts a cardiovascular parameter measurement system worn by a user, according to some embodiments;

FIG. 4b depicts a cardiovascular parameter measurement system worn by a user, according to some embodiments;

FIG. 5 depicts two examples of raw data obtained by a cardiovascular parameter measurement system, according to some embodiments;

FIG. 6 depicts (a) raw data obtained from all sensing elements of a cardiovascular parameter measurement system, (b) isolated motion artifacts obtained from a cardiovascular parameter measurement system, (c) a pressure profile obtained from sensing elements over a blood vessel of a cardiovascular parameter measurement system, and (d) a compressed data series of an oscillometric profile obtained from a cardiovascular parameter measurement system, according to some embodiments;

FIG. 7 depicts refinement and modification of compressed data obtained by a cardiovascular parameter measurement system, according to some embodiments;

FIG. 8 depicts a compressed data obtained by a cardiovascular parameter measurement system operating in a continuous mode, according to some embodiments;

FIG. 9 depicts raw data obtained by a cardiovascular parameter measurement system at six time instances and raw data obtained by a cardiovascular parameter measurement system plotted over a time domain, according to some embodiments; and

FIG. 10 illustrates a computer system that is programmed or otherwise configured to implement methods provided herein, according to some embodiments.

### DETAILED DESCRIPTION

Provided herein are embodiments of a biometric pressure mapping system that provides non-invasive, continuous cardiovascular health parameters measurement, including blood pressure of a wearer, user, individual and/or patient. The biometric pressure mapping system may use a combination of pressure sensing arrays and actuators to produce a dynamic pressure map of the physiological surface, from which the cardiovascular health or physiological measurements are determined.

The system may utilize high spatial resolution, polymeric thin-film sensor arrays inside a system that can apply a controlled pressure and produce a real-time, dynamic pressure distribution map of the skin surface overlying the blood vessel. The dynamic pressure map may be analyzed to determine the physiological signals relevant to the different states of the blood vessel. This analysis may be carried out using two distinct algorithmic approaches namely. A first algorithmic approach may include utilizing dynamic edge-detection and segmentation algorithms on the pressure distribution map data to determine the location and amplitude of the pressure originating from the blood vessel, creating a subtracted pressure map to isolate motion artifacts and external forces exerted by the actuator, and creating a dynamic visualization of the blood vessel characteristic from the pressure map. A second algorithmic approach may comprise performing waveform analysis on the selected pressure elements overlying the blood vessel to determine the consequently the systolic, diastolic and mean arterial pressure values of the test subject.

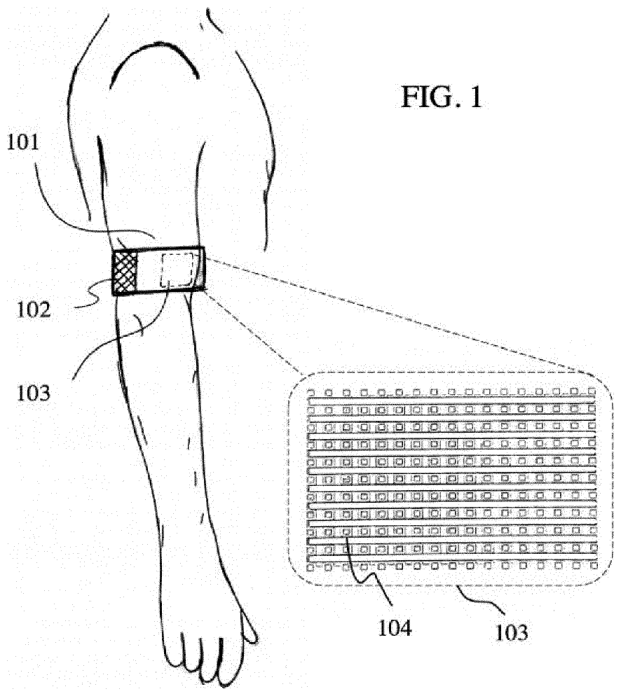

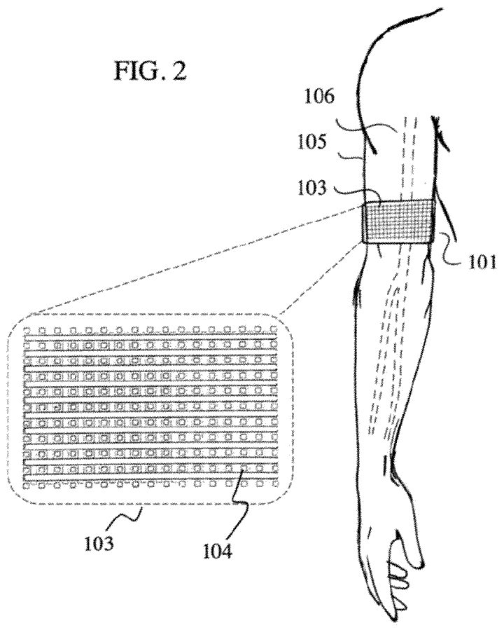

Referring to FIGS. 1 and 2, a support comprising an occlusion sleeve 101 may include a pressure sensing array 103 and an actuation module 102 coupled to the sleeve. The sleeve 101 may be disposed on the upper arm 105 of the user. A breakout view of the pressure sensing array 103 and comprising a plurality of individual sensing elements 104 is shown. The sleeve 101 can apply a controlled pressure on the arm and produce a real-time biometric pressure map of skin surface of a user to which it is applied. A pressure sensor array 103 may be applied such that it overlies the blood vessel 106 under the skin of the user. Each sensing unit may sense changes in pressure of regions or subregions of the skin surface of the user.

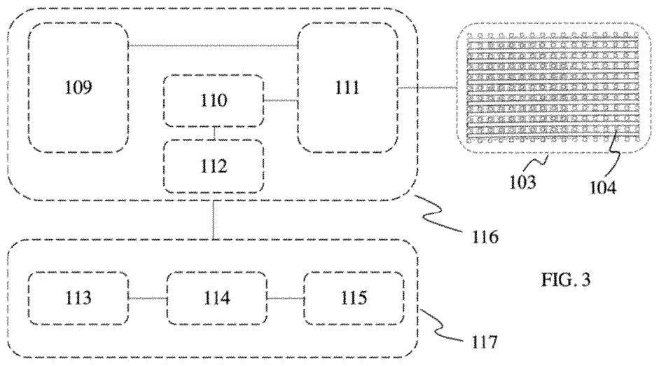

The control module and the constituent modules and parts of the system are depicted as a block diagram in FIG. 3. The pressure sensing array 103 may comprise a plurality of individual sensing elements 104. The pressure sensing array 103 is connected to the data processing module 116, which is connected to data storage and visualization module 117. The data processing module 116. The data processing module may comprise digital signal and image processing module 110, controller unit 111, actuation unit 109, and a biometric processing unit 112. The data storage and visualization module 117 may comprise data storage unit 113, data visualization unit 114 and biometric data display unit 115.

The biometric data display unit may be physical coupled to or provided as part of the sleeve. The display unit may also be detachable from the sleeve. The display unit can receive data from the data processing module to generate the dynamic pressure map by a wired or wireless transmission. The data processing module may transmit data to display a dynamic pressure map on a tertiary device such as a wrist-worn device or phone.

The sensing elements may be comprised of thin-film transducers. The sensing elements may be piezoelectric, piezo resistive, capacitive, electromagnetic, strain gauge pressure sensors, or a combination thereof. The thin-film sensing elements may be comprised of polymers, elastomers, carbon based materials, metals, or a combination thereof.

The control module may act as the interface between the actuator module and the pressure sensor array. The baseline pressure obtained from the pressure sensing array can be used to control the actuator module such that the requisite forces are applied by the actuator on the skin. The system consists of a data processing unit that collects raw data from the pressure sensing array. The data is ideally obtained at a frequency of 100 Hz to 1 kHz, enabling the processing of a dynamic pressure map that includes all physiologically relevant data.

The signal and image processing unit transform the raw data and creates a real-time pressure map of the changes that take place in the surface and the blood vessel underlying the skin. The raw data and the extracted parameters are stored in a data storage module for further processing and data transfer into a centralized server external to the device. The extracted parameters are visualized and displayed in a data visualization unit, which is ideally a built-in display, or an external display interfaced by cables, wires, or wireless means.

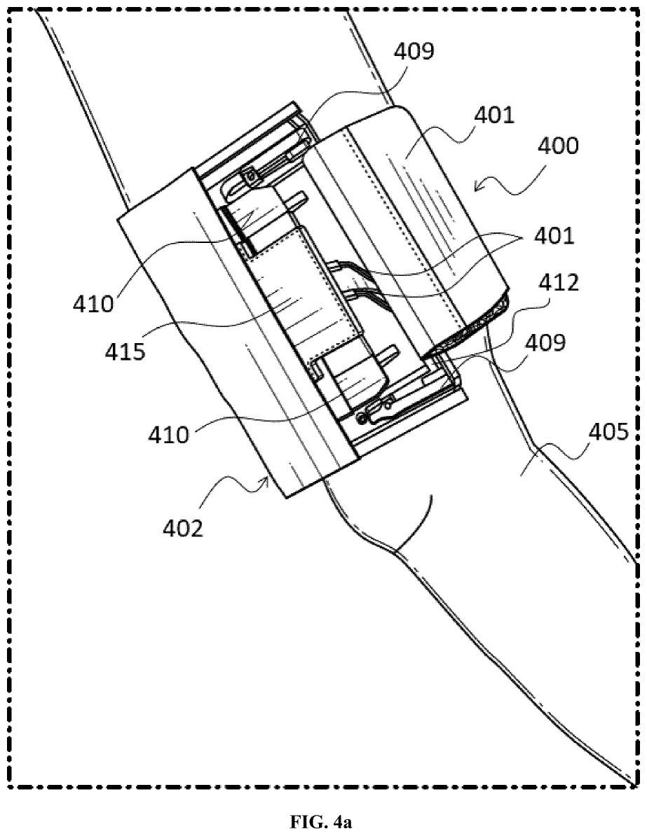

The actuation module as depicted in FIGS. 4A-B may be a mechanical system that applies the requisite pressure, without the need for bulky pneumatic valves, parts and pumps. In some embodiments, the physiological measurement system, comprises a support 400 comprising a sleeve 401, an actuation and data processing unit 402, and the pressure sensing array (e.g. 103 as depicted in FIGS. 1-3), worn over the arm of the user 405. In some embodiments, the pressure sensing array is provided on a surface of the actuation and data processing unit 402 to be placed against a skin surface of a user, wearer, or patient. In some embodiments, the pressure sensing array is provided on an inner surface of the sleeve 401 to be placed against a skin surface of the user. In some embodiments, the pressure sensing array is placed proximal to an artery. In some embodiments, the artery is a brachial artery.

In some embodiments, the actuation and data processing unit 402 comprises one or more arms 409 couples with the sleeve 401 such that the movement of the actuation arm leads to a force being exerted on the arm of the user, resulting in the occlusion of an artery in the arm (e.g. a brachial artery). The pressure is applied by an actuation arm 409 or a combination of arms of the actuator that reduces the effective diameter or circumference of the sleeve so as to reduce the space occupied by the soft tissue and the blood vessel under the sleeve.

In some embodiments, mechanical actuator may comprise of a pair of servo/stepper motors 410 or a bi-axial servo motor interfaced an end of a sleeve that is worn by the user. An actuation arm can be connected to the end of each rotating axel of the servo motor. The arms may be coupled together by metal rod 412 enabling them to move in unison when actuated. The metal rods can be connected in a loop to the sleeve such that the diameter of the sleeve decreases when the servo is actuated. The reduction in diameter causes an increase in pressure on the arm of the user when the sleeve is fastened to the user's arm. The pressure applied on the user's arm is controlled by changing the angle of rotation of the servo motors, wherein an increasing angle may result in a higher pressure. The servo motors can be actuated using a closed-loop control system that takes the mean-pressure value from the sensing array to determine the servo motor angle required to achieve the requisite pressure to partially or completely occlude the blood vessel of the user. The actuation module allows for non-occlusion, partial occlusion, and full occlusion configurations, where the occlusion may refer to occluding a blood vessel proximal or beneath the skin surface being sensed.

In some embodiments, the actuation and data processing unit 402 further comprises an onboard processing unit 415. In some embodiments, wire leads 417 are provided to electrically couple the pressure sensing array to the processing unit 415. In some embodiments, the processing unit receives data from the pressure sensing array and processes the data to provide one or more physiological measurements or physiological monitoring, as disclosed herein. In some embodiments, the processing unit 415 transmits data to an external processing unit. The data may be transmitted via a wired or wireless connection. In some embodiments, the processing unit comprises a non-transitory storage medium comprising software instructions configured to process the data received from the pressure sensor array as disclosed herein. In some embodiments, actuation and data processing unit 402 comprises a battery (not shown) to power to the unit.

Some embodiments of the mechanical actuator include the use of a plurality of low-profile servo motors that are coupled in parallel to enable reduced thickness and weight of the actuation unit. Some embodiments utilize a bank or group of low-power servos with a plurality of coupling arms interfaced with the sleeve such, that the servos are actuated individually or in unison to achieve the pressure required to occlude the blood vessel.

Some embodiments of the actuator mechanism may utilize a windup mechanism that coils up and retracts a part of the sleeve to achieve the reduction in diameter and consequently apply pressure on around the arm of a wearer. This mechanism may use servo motors or low-speed, high-torque motors to produce the winding force and the torque required to occlude the blood vessel.

Alternatively, the mechanical actuation unit may be comprised of combination of a low power actuation mechanism for continuous BP monitoring mode and a high-power actuation mechanism for a calibration mode, wherein a complete oscillometeric waveform and standard systolic blood pressure (SBP) and diastolic blood pressure (DBP) are determined. The low power actuation can be achieved by using small servos or smart material configurations including shape memory alloys (SMA) and electro responsive polymers (ERP). The SMA may be configured as a series of coils or intertwining wires which, when actuated with a current signal, produce the compression forces required to partially/completely occlude the blood vessel to determine continuous BP.

The sleeve may also perform full or partial occlusion of a blood vessel by filling with a fluid, and therefore applying pressure to a portion of a limb of the wearer to which the sleeve is attached.

The system may have two distinct modes of operation, 1) Calibration mode and 2) Continuous measurement mode. The calibration mode may be carried out to obtain the baseline cardiovascular health parameters of the user, to enable accurate estimation of blood pressure, and to prevent measurement drift from reducing the accuracy of the device when it operates continuous measurement mode. The calibration mode measurement may be carried out the first instance the user wears the sleeve, wherein the actuation module applies forces to completely occlude the blood vessel of the user.

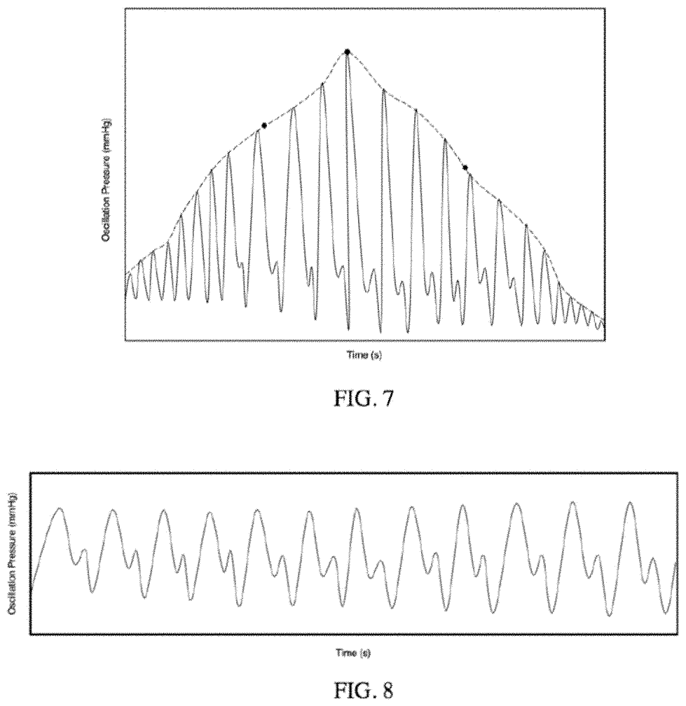

The continuous mode operates with or without minimal constant pressure applied by the actuation module. FIG. 8 shows the low amplitude oscillations measured by the sensing array when the blood vessel is minimally occluded by the actuation unit. The amplitude of the oscillations is used to estimate the blood vessel and the time between two oscillations are used to estimate the heart rate and hart rate variability of the user in a continuous manner. The values are obtained and updated in the data visualization module continuously. The control unit, based on the values obtained from the continuous mode, may determine if a calibration measurement is required to ensure accuracy of the measurement, by using parameters that include time since the last calibration measurement, heart rate of the user, and the state of activity of the user. The system may use a dynamic regime of the two modes to ensure accuracy of the continuous cardiovascular health parameter measurement without the need for external calibration.

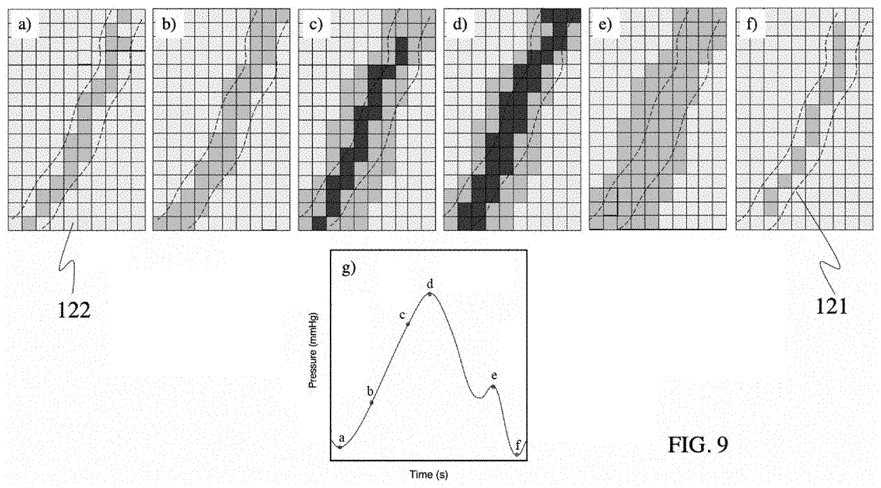

In an oscillometric BP mode, the tightening of the cuff may cause the artery to occlude beyond systolic blood pressure, and corresponding release of the cuff causes the occluded artery to relax/expand. The occlusion, relaxation of the artery, and expansion of the artery is recorded as a pressure map with each pixel of the pressure sensor consisting instantaneous pressure value on the skin surface, as depicted in FIG. 9, data panels a), b), c), d), e), and f). Each pixel may correspond to a particular region or subregion of the skin surface. The recorded raw pressure map is a two-dimensional matrix array of instantaneous pressure values from the pressure sensor. Each frame of the raw pressure map (each of data panels a)-f) of FIG. 9 is a slice of time-domain pressure map signal of the artery surface. The pressure intensity of each pixel has different contributing factors which include the pressure applied by the actuator on the skin, pressure wave generated by the blood vessel, and the pressure generated by the movement of the user. A multi-domain signal analysis on the pressure intensity of each pixel may be used to isolate the different sources of the pressure signal.

The time domain signal for each pixel may be obtained by collecting the instantaneous pressure values corresponding to that pixel from each frame of the raw pressure map. The time domain signal of each pixel may exhibit a characteristic pressure waveform, showing an increase in pressure during tightening of the cuff and a decrease in pressure during release of the cuff. The pressure waveform may contain the pressure oscillations of the artery during its occlusion and relaxation/expansion phases. The time domain signal of each pixel is filtered to remove the noise from the actuation process and the motion artifacts. The filtering process may involve applying finite impulse response (FIR) filters with low pass characteristics to remove the high frequency noise and any direct current (DC) component of the signal. The signal from each pixel is processed using a clustering algorithm that segregates the different pressure components embedded within the raw signal.

Pressure oscillations waveforms or the oscillometric waveform (OMW) can be extracted from the filtered time domain pressure waveform for each pixel after applying the clustering algorithm and baseline correcting the signal. The baseline correction may be performed in two stages: 1. estimating the baseline, and 2. subtracting the estimated baseline from the pressure waveform. The pressure oscillations can have pulse amplitudes in the range of 0.1-4 mmHg. The relative amplitudes of pressure oscillations when they appear on the pressure waveform are smaller and may be hard to extract. In order to extract the pressure oscillations, the filtered time domain pressure waveform may be corrected using two types of baselines. First, a polynomial baseline may be estimated, which follows the pressure waveform containing the pressure oscillations. The pressure waveform is baseline may be corrected by subtracting the estimated polynomial baseline from this signal. The subtracted signal may again be baseline corrected to obtain the OMW. For a second baseline correction, the local minima of the signal may be obtained by analyzing the slope changes (derivatives) of this signal as it contains the crests and troughs. The local minima of this signal may be linearly interpolated to estimate the second baseline. This second baseline may again be subtracted from the signal in order to extract all the pressure oscillations of the OMW.

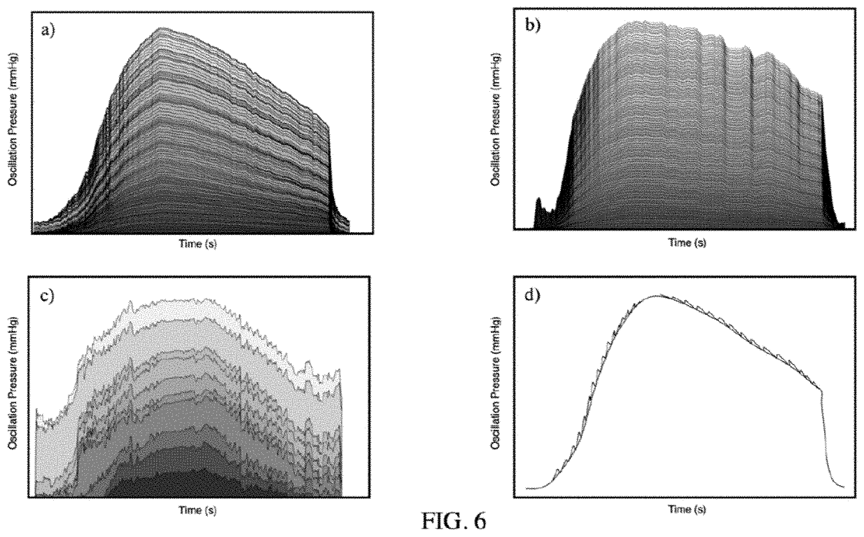

The systolic, diastolic and mean arterial blood pressure of the artery may be estimated from the oscillometric waveform (OMW) signal. For estimating of blood pressures, an envelope may be drawn over the OMW signal, as depicted in FIG. 7. The envelope may be obtained by finding the local maxima of the OMW signal and linearly interpolating these maxima points. The local maxima may be obtained by analyzing the slope changes (derivatives) of OMW signal. In the envelope, time points occurring at specific characteristic ratios called Parameter Identification Point (“PIP”) are found. Pressure measurements on the pressure waveform corresponding to the PIP ratio time points allow obtainment of the systolic, diastolic and mean arterial blood pressures.

The analysis of each pixel of the pressure map in the time and frequency domain may enable the determination of the location and characteristics of the underlying blood vessel beneath the sensor. In oscillometric calibration mode, the systolic blood pressure (SBP) and diastolic blood pressure (DBP) values, that correspond to points in the plane of the sensor, may be isolated after filtering and baseline correction of the raw signal. The subtracted signal may comprise of information about the motion artifacts (which may have different characteristic frequencies and patterns than the physiological signal emanating from the blood vessel). The subtracted signal may be analyzed to determine the state of activity of the user.

The activity state of the user may include whether the user is at rest or in motion. The user may be determined to be in motion if the subtracted signal exceeds a predetermined amplitude threshold and the frequency domain signal falls outside a frequency band determined for the user. In addition, pressure variability pattern for each pixel of the subtracted signal is used to determine the pattern of motion of the user. Specifically, instantaneous signal with frequency above approximately 1 Hz and amplitude above approximately 4 Hz may be classified as motion. Further, signal analysis and filters are used on the signal determined to contain motion artifacts to determine the pattern and nature of motion. If the user is determined to be at rest the system may initiate calibration mode. If the user is determined to be in motion, the continuous mode is used to obtain the pulse amplitude and heart rate which is fed in the estimation algorithm to determine instantaneous blood pressure.

Once the location of the blood vessel is determined from the processed signal, the effect of motion and external pressure may be isolated by carrying out a time, frequency, and space domain clustering analysis of the raw signal obtained from the pixels directly overlying the blood vessel and comparing the pixels that are further away from the blood vessel.

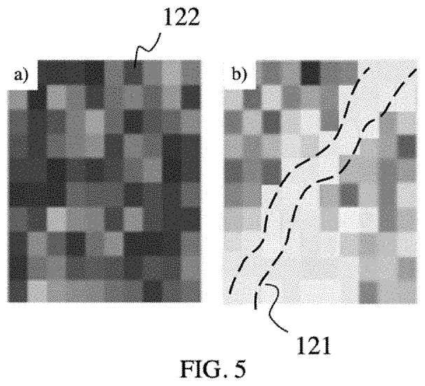

FIG. 6 depicts raw data, included on data panels a), b), c), and d), obtained from the pressure sensing elements where the motion artifacts and the physiological reactions forces are removed to obtain the physiological oscillations from which cardiovascular health parameters including systolic & diastolic blood pressure, heart rate, mean arterial blood pressure, blood vessel viscoelasticity, arterial stiffness are determined. FIG. 6, at data panel a), depicts the raw data obtained from all sensing elements over time as the actuator occludes and releases the blood vessel in the arm of the wearer. FIG. 6, at data panel b), depicts the isolated motion artifacts determined from the pressure distribution map. FIG. 6, at data panel c), depicts the pressure profile of the sensing elements directly overlying the blood vessel, as determined and isolated by the signal and image processing unit. FIG. 6, at data panel d), depicts the compressed data series of the pressure in the form of an oscillometric profile, as typically obtained by an oscillometric blood pressure measurement system.

The blood pressure may be determined using a fixed ratio and/or adaptive ratio methods. The arterial stiffness and the viscoelastic properties of the blood vessel may be determined by analyzing the relationship between pressure variations and the displacement of the blood vessel over time. These parameters may be used to determine the cardiovascular fitness and the health of the user.

The blood pressure measurement from the biometric pressure map is obtained by using oscillometric and compression analysis principles from the subtracted pressure map encoding the information about the cardiovascular health parameters as depicted in FIGS. 6-8. FIG. 6 depicts the processing and filtering of raw sensing array data to obtain a single oscillometric waveform that is identical to the raw data obtained from an oscillometric cuff-based BP monitor. FIG. 7 shows the extracted waveform and an oscillometric envelope obtained from the raw data as shown in FIG. 6, at data panel d). The oscillometric profile enables the determination of the systolic, diastolic and mean arterial pressure of the user by fixed ratio and/or adaptive ratio methods. The cardiovascular health parameters obtained using this approach is used as the baseline values for the individual user.

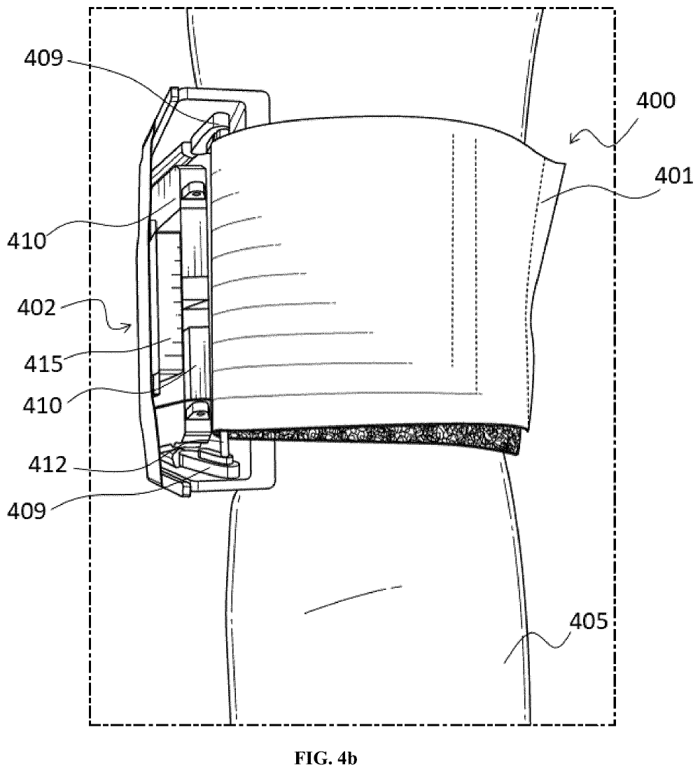

The sensing array may obtain the pressure variations to create a real-time, dynamic pressure map of the surface as shown in FIGS. 5 and 9. Each pixel 122 in may represent the pressure profile at that point on the skin. Each pixel may represent an individual pressure sensing element (104 as depicted in FIGS. 1-3). The dynamic pressure map can effectively produce a three-dimensional video of the surface, with the intensity changes representing the pressure variations of each sensing element in the array. This pressure profile and the pressure intensity map may be processed by a signal and image processing module to extract the location of the blood vessel 121 and biometric oscillations (FIGS. 6, 7, 8, and 9) using an edge detection and oscillation extraction algorithm. The pressure map may be further analyzed to develop models that accurately predict the sensing elements that lie closest to the artery being monitored. To achieve this, k-nearest neighbor classification and edge detection algorithms may be utilized. The edge and line detection are carried using gradient operators (e.g. Prewitt, Sobel, Laplacian, etc.) on individual time frames of the pressure map can enable dynamic detection of the blood vessel interface at different points in time.

The data obtained from the processing module may be used to separate the pressure variations caused due to the physiological reaction forces exerted by the blood vessel from the external pressure applied by the actuator and pressure perturbations due to motion artifacts caused by the user, as described supra, to create a subtracted pressure map as shown in FIGS. 5 and 9. The subtracted pressure map may provide real-time changes in the blood flow within the blood vessel, mechanical oscillations due to the flow, and the mechanical properties of the blood vessel.

### I. Computer Systems

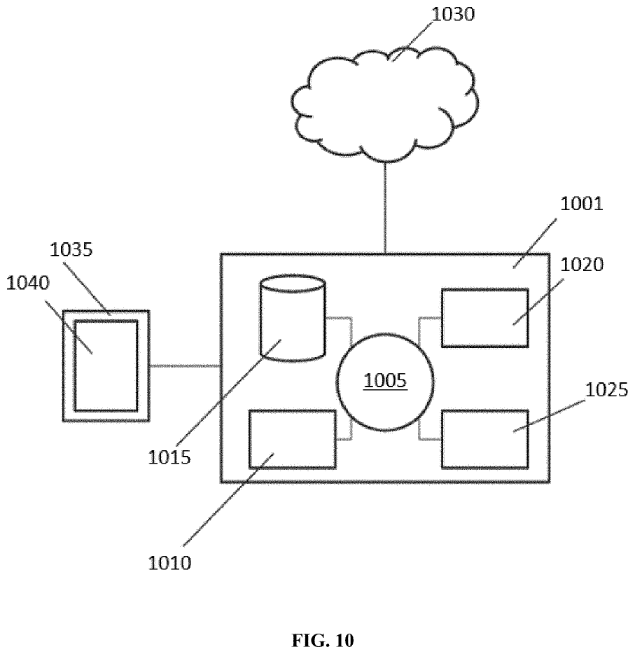

The present disclosure provides computer systems that are programmed to implement methods of the disclosure, according to some embodiments. FIG. 10 shows a computer system 1001 that is programmed or otherwise configured to, for example, implement the methods disclosed herein. The computer system 1001 can be an electronic device of a user or a computer system that is remotely located with respect to the electronic device. The electronic device can be a mobile electronic device.

The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries, and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user (e.g., a smartphone, a laptop computer). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine-readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040. Examples of UI's include, without limitation, a graphical user interface (GUI), a mobile device application, and web-based user interface.

### II. Currently Preferred Embodiment

1. In one currently preferred embodiment, the invention provides a physiological measurement system comprising: a support configured to engage a wearer; and a sensor array coupled to the support, the sensor array configured to sense one or more pressure changes on a skin surface of the wearer and determine one or more physiological parameters of the wearer based on the one or more sensed pressure changes
2. The system of paragraph 1, further comprising a data processing module configured to generate a dynamic pressure map of the skin surface based on the one or more sensed pressure changes.
3. The system of paragraph 2, further comprising a biometric display unit to display the dynamic pressure map.
4. The system of paragraph 3, wherein the biometric display unit is physically coupled to the support.
5. The system of paragraph 3 or 4, wherein the data processing module is configured to wirelessly transmit the dynamic pressure map to the biometric display unit.
6. The system of any one of paragraphs 1 to 5, wherein at least one of the one or more sensed pressure changes is associated with a blood vessel proximal to the skin surface of the wearer.
7. The system of paragraph 6, wherein at least one of the one or more physiological parameters are parameters is associated with the blood vessel.
8. The system of paragraph 6 or 7, wherein the one or more physiological parameters comprise a systolic blood pressure, a diastolic blood pressure, a mean blood pressure, a heart rate, a blood vessel viscoelasticity, an arterial stiffness, or a combination thereof.
9. The system of any one of paragraphs 6 to 8, wherein the support is configured to engage a portion of a limb of the wearer, the support configurable in:

a non-occlusion configuration; and

an occlusion configuration for occluding the blood vessel.

10. The system of paragraph 9, wherein the support is further configurable in a partial occlusion configuration for partially occluding the blood vessel.
11. The system of paragraph 9 or 10, wherein a mechanism to change the configuration of the support comprises an actuator mechanism, a mechanical actuation mechanism, a fluid actuation mechanism, or a combination thereof.
12. The system of any one of paragraphs 9 to 11, wherein the support comprises a sleeve, wherein the sleeve engages the portion of the limb of the wearer at an effective diameter, and wherein the effective diameter is reduced to occlude the blood vessel.
13. The system of any one of paragraphs 9 to 12, wherein the system is operable in a calibration mode, wherein operation in the calibration mode configures the support in the occlusion configuration for calibrating baseline parameters the one or more physiological parameters.
14. The system of any one of paragraphs 10 to 13, wherein the system is operable in a continuous monitoring mode, wherein operation in the continuous mode configures the support in the partial occlusion configuration for monitoring the one or more physiological parameters
15. The system of any one of paragraphs 1 to 14, wherein the sensor array is comprised of a plurality of sensing elements.
16. The system of any one of paragraphs 1 to 15, wherein the plurality of sensing elements comprises thin-film transducers.
17. The system of paragraph 16, wherein the thin-film transducers are polymeric thin-film transducers.
18. In one currently preferred embodiment, the invention provides a method of measuring one or more physiological parameters, comprising: measuring one or more changes in pressure on one or more a regions of a skin surface of a wearer; and generating a dynamic pressure map of the skin surface of the wearer based on the measured one or more changes in pressure of the skin surface.
19. The method of paragraph 18, wherein the step of measuring one or more changes in pressure on the skin surface of the wearer further comprises measuring physiological reaction forces on the skin surface of the wearer.
20. The method of paragraph 19, wherein the physiological reaction forces are oscillometric pressure waves generated by a blood vessel proximal to the skin surface of the wearer.
21. The method of any one of paragraphs 18 to 20, further comprising a step of determining a location of a blood vessel proximal to the skin surface of the wearer.
22. The method of paragraph 21, further comprising isolating one or more changes in pressure of the skin surface at the location of the blood vessel.
23. The method of paragraph 22, wherein the step of isolating the one or more changes in pressure at the location of the blood vessel comprises carrying out a time, frequency, and space domain clustering analysis of a comparison of the changes in pressure at the location of the blood vessel relative to one or more changes in pressure on the skin surface of the wearer at a location away from the blood vessel.
24. The method of any one of paragraphs 20 to 23, further comprising steps of occluding the blood vessel; and calibrating the one or more measured pressure changes of the skin surface.
25. The method of paragraph 24, wherein the step of occluding the blood vessel comprises fully occluding the blood vessel.
26. The method of any one of paragraphs 20 to 25, further comprising steps of partially occluding the blood vessel; and monitoring for one or more changes in pressure of the skin surface.
27. The method of any one of paragraphs 18 to 26, wherein the one or more physiological parameters comprise a systolic blood pressure, a diastolic blood pressure, a mean blood pressure, a heart rate, a blood vessel viscoelasticity, an arterial stiffness, or a combination thereof.
28. In one currently preferred embodiment, the invention provides a wearable physiological measurement device, comprising: a support configured to engage a portion of a limb of a wearer; a sensor array coupled to the support and configured to sense one or more pressure changes on a skin surface of the wearer; and a data processing module configured to generate a dynamic pressure map of the skin surface based on the one or more sensed pressure changes on the skin surface, wherein one or more physiological parameters of the wearer are derived from the dynamic pressure map.
29. The device of paragraph 28, wherein the data processing module is configured to transmit the dynamic pressure map to a biometric display unit.
30. The device of paragraph 29, wherein the biometric display unit is physically coupled to the support.
31. The device of any one of paragraphs 28 to 30, wherein at least one of the one or more sensed pressure changes is associated with a blood vessel proximal to the skin surface of the wearer.
32. The device of paragraph 31, wherein the at least one of the one or more physiological parameters is associated with the blood vessel.
33. The device of paragraph 31 or 32, wherein the support is configurable in: a non-occlusion configuration; and an occlusion configuration for occluding the blood vessel.
34. The device of paragraph 33, wherein the support is further configurable in a partial occlusion configuration for partially occluding the blood vessel.
35. The device of any one of paragraphs 33 or 33, wherein a mechanism to change the configuration of the support comprises an actuator mechanism, a mechanical actuation mechanism, a fluid actuation mechanism, or a combination thereof.
36. The device of any one of paragraphs 33 to 35, wherein the support comprises a sleeve, wherein the sleeve engages the portion of the limb of the wearer at an effective diameter, and wherein the effective diameter is reduced to occlude the blood vessel.
37. The device of any one of paragraphs 33 to 36, wherein the device is operable in a calibration mode, wherein operation in the calibration mode configures the support in the occlusion configuration for calibrating baseline parameters for the one or more physiological parameters.
38. The device of any one of paragraphs 33 to 37, wherein the device is operable in a calibration mode, wherein operation in the calibration mode configures the support in the occlusion configuration for monitoring the one or more physiological parameters.
39. The device of any one of paragraphs 28 to 38, wherein the sensor array is comprised of a plurality of sensing elements.
40. The device of paragraph 39, wherein the plurality of sensing elements comprises thin-film transducers.
41. The device of paragraph 39, wherein the thin-film transducers are polymeric thin-film transducers.
42. The device of any of paragraphs 28 to 41, wherein the one or more physiological parameters comprise a systolic blood pressure, a diastolic blood pressure, a mean blood pressure, a heart rate, a blood vessel viscoelasticity, an arterial stiffness, or a combination thereof.
43. In one currently preferred embodiment, the invention provides a wearable blood pressure measurement device, comprising: a sleeve configured to engage a portion of a limb of a wearer; a sensor array coupled to the sleeve and configured to sense one or more changes in pressure on a skin surface associated with a blood vessel; and a data processing module configured to generate a dynamic pressure map of the blood vessel based on the one or more sensed pressure changes, wherein a blood pressure of the wearer is determined from the dynamic pressure map.
44. The device of paragraph 43, wherein the data processing module is configured to transmit the dynamic pressure map to a biometric display unit.
45. The device of paragraph 44, wherein the biometric display unit is physically coupled to the sleeve.
46. The device of any one of paragraphs 43 to 45, wherein the sleeve engages the portion of the limb of the wearer at an effective diameter, the sleeve configurable in: a non-occlusion configuration; and an occlusion configuration for occluding the blood vessel.
47. The device of paragraph 46, wherein the sleeve further comprises a partial occlusion configuration for partially occluding the blood vessel.
48. The device of paragraph 46 or 47, wherein the effective diameter of the sleeve is reduced to occlude the blood vessel.
49. The device of paragraph 48, wherein a mechanism to reduce the effective diameter of the sleeve comprises an actuator mechanism, a fluid actuation mechanism, or a combination thereof.
50. The device of any one of paragraphs 46 to 49, wherein the device is operable in a calibration mode, wherein operation in the calibration mode configures the sleeve in the occlusion configuration for calibrating baseline parameters the blood pressure of the wearer.
51. The device of any one of paragraphs 47 to 50, wherein the device is operable in a continuous monitoring mode, wherein operation in the calibration mode configures the sleeve in the partial occlusion configuration for monitoring the blood pressure of the wearer.
52. The device of any one of paragraphs 43 to 51, wherein the sensor array is comprised of a plurality of sensing elements.
53. The device of paragraph 52, wherein the plurality of sensing elements comprises thin-film transducers.
54. The device of paragraph 53, wherein the thin-film transducers are polymeric thin-film transducers.
55. The device of any of paragraphs 43 to 54, wherein the blood pressure comprises a systolic blood pressure, a diastolic blood pressure, a mean blood pressure, or a combination thereof.
56. The device of any one of paragraphs 43 to 55, wherein a heart rate, a blood vessel viscoelasticity, an arterial stiffness, or a combination thereof is further determined from the dynamic pressure map.

### III. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “about” a number refers to that number plus or minus 10% of that number. The term “about” a range refers to that range minus 10% of its lowest value and plus 10% of its greatest value.

The section headings used herein are for organizational purposes only and are not to be construed as limiting the subject matter described.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-17. (canceled)

18. A method of measuring one or more physiological parameters, comprising:
measuring one or more changes in pressure on one or more a regions of a skin surface of a wearer; and
generating a dynamic pressure map of the skin surface of the wearer based on the measured one or more changes in pressure of the skin surface.

19. The method of claim 18, wherein the measuring one or more changes in pressure on the skin surface of the wearer further comprises measuring physiological reaction forces on the skin surface of the wearer.

20. The method of claim 19, wherein the physiological reaction forces are oscillometric pressure waves generated by a blood vessel proximal to the skin surface of the wearer.

21. The method of claim 18, further comprising:
determining a location of a blood vessel proximal to the skin surface of the wearer.

22. (canceled)

23. The method of claim 21, further comprising:
isolating one or more changes in pressure of the skin surface at the location of the blood vessel,
wherein isolating the one or more changes in pressure of the skin surface at the location of the blood vessel comprises carrying out a time, frequency, and space domain clustering analysis of a comparison of the changes in pressure at the location of the blood vessel relative to one or more changes in pressure on the skin surface of the wearer at a location away from the blood vessel.

24. The method of claim 20, further comprising:
occluding the blood vessel; and
calibrating the one or more measured pressure changes of the skin surface.

25. (canceled)

26. (canceled)

27. The method of claim 18, further comprising:
determining one or more physiological parameters of the wearer based on the measured one or more changes in pressure of the skin surface, wherein the one or more physiological parameters comprise a systolic blood pressure, a diastolic blood pressure, a mean blood pressure, a heart rate, a blood vessel viscoelasticity, an arterial stiffness, or a combination thereof.

28. A wearable physiological measurement device, comprising:
a support configured to engage a portion of a limb of a wearer;
a sensor array coupled to the support and configured to sense one or more pressure changes on a skin surface of the wearer; and
a data processing module configured to generate a dynamic pressure map of the skin surface based on the one or more sensed pressure changes on the skin surface,
wherein one or more physiological parameters of the wearer are derived from the dynamic pressure map.

29. The device of claim 28, wherein the data processing module is configured to transmit the dynamic pressure map to a biometric display unit.

30. (canceled)

31. The device of claim 28, wherein at least one of the one or more sensed pressure changes is associated with one or more physiological parameters of a blood vessel proximal to the skin surface of the wearer.

32. (canceled)

33. The device of claim 31, wherein the support is configurable in a configuration of either:
a non-occlusion configuration; or
an occlusion configuration for occluding the blood vessel.

34. (canceled)

35. The device of claim 33, wherein a mechanism to change the configuration of the support comprises an actuator mechanism, a mechanical actuation mechanism, a fluid actuation mechanism, or a combination thereof.

36. (canceled)

37. The device of claim 33, wherein the device is operable in a calibration mode, and wherein operation in the calibration mode configures the support in the occlusion configuration for calibrating baseline parameters for the one or more physiological parameters.

38. The device of claim 33, wherein the device is operable in a calibration mode, and wherein operation in the calibration mode configures the support in the occlusion configuration for monitoring the one or more physiological parameters.

39. The device of claim 28, wherein the sensor array is comprised of a plurality of sensing elements, wherein the plurality of sensing elements comprises polymeric thin-film transducers.

40. (canceled)

41. (canceled)

42. The device of claim 28, wherein the one or more physiological parameters comprise a systolic blood pressure, a diastolic blood pressure, a mean blood pressure, a heart rate, a blood vessel viscoelasticity, an arterial stiffness, or a combination thereof.

43. A wearable blood pressure measurement device, comprising:
a sleeve configured to engage a portion of a limb of a wearer;
a sensor array coupled to the sleeve and configured to sense one or more changes in pressure on a skin surface associated with a blood vessel; and
a data processing module configured to generate a dynamic pressure map of the blood vessel based on the one or more sensed pressure changes,
wherein a blood pressure of the wearer is determined from the dynamic pressure map.

44. The device of claim 43, wherein the data processing module is configured to transmit the dynamic pressure map to a biometric display unit, wherein the biometric display unit is physically coupled to the sleeve.

45. (canceled)

46. The device of claim 43, wherein the sleeve engages the portion of the limb of the wearer at an effective diameter, the sleeve configurable in either:
a non-occlusion configuration; or
an occlusion configuration for occluding the blood vessel.

47. The device of claim 46, wherein the sleeve further comprises a partial occlusion configuration for partially occluding the blood vessel.

48. The device of claim 46, wherein the effective diameter of the sleeve is reduced to occlude the blood vessel.

49. The device of claim 48, wherein a mechanism to reduce the effective diameter of the sleeve comprises an actuator mechanism, a fluid actuation mechanism, or a combination thereof.

50. The device of claim 46, wherein the device is operable in a calibration mode, wherein operation in the calibration mode configures the sleeve in the occlusion configuration for calibrating baseline parameters the blood pressure of the wearer.

51. The device of claim 47, wherein the device is operable in a continuous monitoring mode, wherein operation in the calibration mode configures the sleeve in the partial occlusion configuration for monitoring the blood pressure of the wearer.

52. The device of claim 43, wherein the sensor array is comprised of a plurality of sensing elements.

53. The device of claim 52, wherein the plurality of sensing elements comprises thin-film transducers.

54. The device of claim 53, wherein the thin-film transducers are polymeric thin-film transducers.

55. The device of claim 43, wherein the blood pressure comprises a systolic blood pressure, a diastolic blood pressure, a mean blood pressure, a heart rate, a blood vessel viscoelasticity, an arterial stiffness, or a combination thereof.

56. (canceled)
